(19) United States
(12) Patent Application Publication
(10) Pub. No.: US 2005/0221481 A1
(43) Pub. Date: Oct. 6, 2005

(54) Amplification of T cells from human cord blood in serum-deprived culture stimulated with stem cell factor, interleukin-7 and interleukin-2

(75) Inventors: Giovanni Migliaccio, Tivoli (IT); Anna Rita Franco Migliaccio, Tivoli (IT); Massimo Sanchez, Roma (IT); Elena Alfani, Roma (IT)

Correspondence Address: ROTHWELL, FIGG, ERNST & MANBECK, P.C., 1425 K STREET, N.W., SUITE 800, WASHINGTON, DC, 20005, US

(73) Assignee: ISTITUTO SUPERIORE DI SANITA', Roma (IT)

(21) Appl. No.: 10/812,361

(22) Filed: Mar. 30, 2004

(51) Int. Cl. C12N 5/08

(52) U.S. Cl. 435/372

(57) ABSTRACT

The present invention relates to a method of ex vivo amplification of neonatal T cells from umbilical cord blood which comprises obtaining light density mononuclear cells from a sample of umbilical cord blood and then culturing said light density mononuclear cells in a serum-deprived culture medium supplemented with various cytokine combinations. Particularly, there are reported the effects exerted by cytokine combinations including stem cell factor (SCF), interleukin-7, interleukin-4 and interleukin-2, on the amplification of T cells from cord blood mononuclear cells cultured for 10-11 days under serum-deprived conditions. Of all the combinations investigated, SCF plus interleukin-7 sustained the best fold increase (FI) of total nucleated cells (FI=6.4±1.17), amplifying preferentially CD4⁺ over CD8⁺ T cell subsets (FI=4.72±0.79 vs 2.73±1.2, respectively, p<0.05). The addition of interleukin-2 to this combination did not significantly increase the total number of cells generated (FI=7.4±2.27) but allowed preferential amplification of CD8⁺ over CD4⁺ T cells (FI=6.04±0.14 vs 1.67±0.6, respectively, p<0.05). Single strand conformation polymorphism analysis of the T-cell receptor V_{β}-chain rearrangements expressed by the expanded T cells indicated that the complexity of the T-cell repertoire had increased after 10 days of culture in the presence of SCF and IL-7. Interestingly, a modest expansion (FI=8.67±1.5) of myeloid progenitor cells was also observed in these cultures. These results indicate that it is possible, by modulating the cytokines added to the cultures, to expand specific T cell subsets for adoptive immunotherapy without lousing myeloid progenitor cells necessary for neutrophil recovery after cord blood transplantation.

### BACKGROUND OF THE INVENTION

The use of cord blood (CB) as an alternative source of hemopoietic stem cells for allo-transplantation of pediatric patients is steadily increasing (1). These transplants experience a rate of engraftment similar to that reported for allogeneic adult marrow transplants, but their follow-up is characterized by reduced incidence of graft-versus-host disease (GVHD) (2). The reduced GVHD has been explained by the low immune-reactivity of neonatal circulating T lymphocytes (3-5) and may result in reduced graft-versus-leukemia (GVL) effect as well.

Donor lymphocyte infusion (DLI) is a supportive therapy currently pursued to increase GVL after bone marrow transplantation (6, 7). Its clinical application to cord blood transplantation is limited by the reduced volume of CB available as a graft from a single delivery. This problem, however, could be solved by using as DLI ex-vivo expanded T (CD3⁺) cells obtained from a portion of the CB unit (8) and pre-clinical studies are under way to analyze the GVL effect of ex vivo-amplified neonatal T-cells in leukemic and cancer patients (8). Most of the ex vivo manipulation and amplification of T cells realized up to now (9-11) are sustained by a combination of IL-2 with either anti-CD3 or anti-CD28 (8, 9, 11) and the culture conditions include serum, either fetal bovine or human (9,12). Furthermore, none of these studies have investigated whether the increases in T cell number are associated with increases in T cell clonality.

In addition to anti-CD3 and CD28 antibodies, several growth factors, such as interleukin (IL)-2, IL-4 , IL-7, are known to regulate the life span and/or the activation of T-lymphocytes in vitro and in vivo (13-16). The use of these growth factors for ex-vivo amplification of neonatal T cells under serum-deprived conditions has not been explored as yet. This study analyses the effects of SCF, in combination with the T-cell-specific growth factors mentioned above, on ex vivo expansion of T cells, both in terms of total cell number and of total number of T cell clones, in serum-deprived cultures of light density CB cells. Two growth factor combinations (SCF+IL-7 and SCF+IL-7+IL-2) were found to induce levels of T cell amplifications similar to those previously reported to be sustained by anti-CD3 plus IL-2 (8, 9). SCF plus IL-7 were found to stimulate preferentially amplification of CD4⁺ T cells while SCF+IL-7+IL-2 increased preferentially the number of CD8⁺ T cells. Interestingly, expansion of myeloid progenitor cells, the injection of which could result in reduced neutropenia after CB transplants (17), was also observed in these cultures. Therefore, ex vivo culture of a fraction of a CB graft with combinations of T cell specific growth factors could amplify at the same time both T cells, for supportive immunotherapy while maintaining the number of progenitor cells, to accelerate the speed of the engraftment.

### SUMMARY OF THE INVENTION

It is here characterized the T cells expansion obtained in serum-deprived cultures of neonatal hemopoietic cells. To reproduce conditions that most likely will be used in clinical settings, the culture were seeded with the mononuclear fraction of CB, although in selected experiments purified neonatal T cells were cultured under the same conditions for comparison. The cultures were stimulated with the early multi-lineage cytokine SCF in combination with either IL-3, a factor know to stimulate proliferation of CD34⁺ cells purified from either neonatal (25-27) or adult (33) specimens, or with IL-7, IL-4 and IL-2, cytokines known for their activity on lymphoid cells (34). IL-7 provides a non-redundant signals for T- and B-cells development and is produced by the same stromal cells that produce SCF in the fetal liver, in the adult bone marrow and in the thymus (14, 35), where the highest levels of IL-7 production occur in vivo (36). On the other hand, although mouse models have demonstrated that IL-2 and IL-4 are dispensable for appropriate lymphoid development in vivo (15, 16), these growth factors, that are produced mainly by specific T cell subsets upon activation (37, 38), play important roles in regulating T cell function in vitro.

In contrast with what reported for purified CD34⁺ cells (25-27, 33), SCF+IL-3 did not increase the number of total nucleated cells observed after 10-12 days in serum-deprived culture of CB-LDC and induced only a modest (5-fold) increase in the total number of CFC. Such modest increases are likely to be due to the fact that, in contrast with the purified cells, LDC contains a high proportion of differentiated cells that die out during the culture masking the output of newly differentiated cells from the progenitor cell compartment. As expected, most of the cells grown in the presence of this growth factor combination fall outside the lymphocyte gate and were identified as myeloid (CD33⁺) cells. These results unveil a lineage species-specificity for the action of these two growth factors: in fact, SCF and IL-3 have been reported to induce T cell differentiation in cultures of murine marrow cells (29, 39) but are a poor stimulus for T cell proliferation in cultures of human cells where they sustain preferentially amplification of myeloid cells (25-27, 33 and this manuscript).

In agreement with the biological activity briefly summarized above, IL-7 was the only cytokine found to synergize with SCF in sustaining amplification of T cells. In fact, the combination of SCF+IL-7 increased the total number of cells observed after 10 days of culture more than any other growth factor combination analyzed (Table 1). Such an increase in total cell number was associated with increases in cells of the lymphoid compartment (Table 2). Both CD4⁺ and CD8⁺ cells increased after SCF+IL-7 stimulation, although CD4⁺ cells were preferentially amplified (Table 2). Analysis of the TCR β chain gene rearrangements showed that the increases in T cell number were associated with increases in T-cell clonality. Therefore, the T cells amplification observed in these cultures was not only due to amplification of clones already significantly represented in the original CB-LDC population but new clones, that were non present at day 0, became also manifested with time in culture. The dynamic pattern of expression of the TCR β-chain rearrangements observed in the time course analysis indicates that different individual T-lymphocyte clones become prevalent at any given time in culture. It is possible that new T cell clones are continuously generated ex-novo in culture of CB-LDC from precursor/progenitor cells and that those clones disappear after an initial amplification because lacking a still to be identified survival stimulus. Support for this interpretation is provided by reports that stem/progenitor cells differentiate into mature CD4⁺ or CD8⁺ T cells in culture not only in the presence of accessory cells of thymic origin (28, 30, 40) but also in the presence of certain cell lines (41) and of accessory cells present in the murine marrow (42) and in human neonatal blood (19). However, the hypothesis that in the cultures described here, T cells derive by differentiation of an early compartment is in contrast with the observation that the frequencies of the T cell precursors (CD7⁺/CD2⁻ and CD4⁺/CD8⁺) were found to be only modestly increased. Alternatively, the dynamic pattern of rearrangements observed in these cultures could simply reflects stochastic variations in the proliferation of individual T cell clones. To directly exclude this possibility, purified neonatal CD4⁺ and CD8⁺ T cells were cultured under the same conditions that had been found to sustain the best proliferation of T cells from CB-LDC. Purified T cells proliferated less efficiently and expressed a lower number of TCR Vβ gene rearrangements than CB-LDC. Furthermore, while in culture of CB-LDC stimulated with SCF+IL-7, CD4⁺ cells increased preferentially over CD8⁺ cells (Table 2), the two cell populations had similar survival when cultured as purified cells.

IL-2 had no biological activity when used directly in combination with SCF but exerted very distinct effects when either IL-4 or IL-7 was also present in the culture system. Its addition to the combination of SCF+IL-7 suppressed amplification of CD4⁺ T cells, favored the amplification of CD8⁺ T cells and did not affect the number of CFC generated over time. On the other hand, when used in combination with SCF+IL-4, it had no effect on the T cell compartments but completely abrogated the amplification of progenitor cells induced by this growth factor combination. Therefore, the cellular levels of its action (progenitor cells vs differentiated T cells) and the type (inhibitory or stimulatory) were linked to whether IL-4 or IL-7 were also present. The receptor complexes for IL-2, IL-4 and IL-7 display a similar organization characterized by the association of the cytokine-specific binding subunit with a signaling β subunit common to all of the three growth factors (43-46). The results presented here indicate that the binding subunits may establish specific competitions for the signaling common subunit in different cell populations (i.e. progenitor cells and differentiated T cells). It is possible that at the progenitor levels, the IL-2-binding subunit competes with the IL-4-binding subunit for activation of the common subunit while at the T cell levels, the IL-2- and IL-7-binding subunits synergize in activating the common subunit.

In conclusion, we describe that either SCF+IL-7 or SCF+IL-7+IL-2 sustain in vitro amplification of T cells at levels comparable to those reported to be induced by the combination of IL-2 and anti-CD3 antibody (8, 9, 11). The increases in T cell number were associated with a considerable increase in the complexity of the TCR repertoire expressed by the cultured T cells. Although the levels of amplifications achieved in this paper were sensibly lower than those induced by the mitogens concanavalin A and mezerein (10), it is debatable whether mitogens will be used for clinical purposes in the near future. Furthermore, in contrast to all the conditions for T cell amplification established up to now, stimulation of CB-LDC with lymphoid-specific growth factor combinations sustained not only amplification of T cells but also that of progenitor cells. Therefore, amplification of T cells for supportive immunotherapy can occur under the same culture conditions that maintain progenitor cells for myeloid engraftment.

### MATERIAL AND METHODS

Cord blood samples and Cell Purification. Umbilical cord blood (CB) was obtained at the time of delivery from uncomplicated pregnancies with previous written informed consent from the mother. Light density mononuclear cells (LDC) were isolated from fresh specimens by density-cut separation (p<1.077) (Ficoll-Paque; Amersham-Pharmacia Biotech, Uppsala, Sweden) and either processed directly or cryopreserved in Hank's balanced salt solution supplemented with 10% (v/v) DMSO and 50% (v/v) deionized bovine serum albumin (Sigma, St. Louis, Mo.) for later use as described (18).

Cell Culture. CB-LDC (10⁵/ml) were expanded for up to 10-12 days in liquid cultures stimulated with SCF, IL-2 and IL-4 (10 ng/ml, 100 ng/ml and 100 ng/ml, respectively, a gifts from Amgen, Thousand Oaks, Calif.), IL-3 (10 U/ml, a gift of Genetic Institute, Boston, Mass.), IL-7 (10 ng/ml, R&D Systems, Minneapolis, Minn.), under serum-deprived conditions as described (19). The cultures were demipopulated and fresh culture medium and growth factors added as required to keep cell concentration below 6×10⁵ cells/ml. The number of myeloid progenitor cells (CFC) was evaluated in cultures made semisolid with methylcellulose (0.8% wt/v) dissolved in Iscove's modified Dulbecco's medium and stimulated with a mixture of growth factors including IL-3 (10 u/ml), SCF (10 ng/ml), erythropoietin (Epo, 1.5 u/ml, Epoetina α, Dompè Biotec, Milan, Italy), granulocyte colony-stimulating factor (G-CSF, 10 ng/ml, Filgrastim, Dompè Biotec) and granulocyte macrophage- colony-stimulating factor (GM-CSF, 10 ng/ml, Molgramostim, Sandoz, Milan, Italy) as previously described (20). The colonies derived from burst forming units, erythroid (BFU-E), colony forming units, granulo-monocytic (CFU-GM) and mixed, erythroid and myeloid (CFU-mix) progenitor cells were scored according to standard criteria after 14 days of incubation in a fully humidified CO₂/O₂ (5% each) monitored incubator.

Immunophenotyping by flow cytometry. Cells were washed twice in phosphate buffer saline and incubated (1-5×10⁵ cells per tube) at 4° C. for 30 min with antibodies against CD3, CD4, CD7, CD8, CD45 (all from Sigma), CD45RA, CD45RO (both from Beckman Coulter Inc, Fulleton, Calif., USA), CD19, CD34 (both from Becton Dickinson, Franklin Lakes, N.J., USA), CD2 and CD33 (PharMingen, San Diego, Calif., USA) all labeled with appropriate fluorochromes. Aliquots of the cells were also incubated with appropriate isotype matched antibodies as control for non specific binding. Two- and three-colour cytofluorimetric analysis and cell sorting of CD4 and CD8 single positive cells were performed with a Coulter Elite ESP Cell Sorter (Coulter, Miami, Fla., USA).

Mixed Lymphocyte Reaction (MRL). Mixed lymphocyte cultures were obtained incubating LDC cells from either adult peripheral blood (PB) or from CB, or lymphocytes obtained after culturing CB-LDC for 10 days in the presence of SCF and IL-7 (10⁵ cells per well per three replicate wells) in the presence of irradiated autologous LDC (either from PB or CB, as appropriate) or irradiated allogeneic PB-LDC (2×10⁵ cells per well in all the cases)(21). The cells used as stimulator had been irradiated at 1000 rad with the GAMMA CELL 220 (Atomic Energy Canada Ltd, Toronto, Canada). Each well contained 0.2 ml of Iscove's modified Dilbecco's medium (IMDM) supplemented with 10% (v/v) human AB serum. After 6 days of incubation at 37° C. in 5% CO₂, the plates were pulsed for 16 hrs. with Methyl-³H-Thymidine ([³H]TdR, 0.5 μCi/well, specific activity 5 Ci/mmol, Amersham Pharmacia Biotech Italia, Cologno Monzese, Italy). The cells contained in each well were then deposited onto glass fiber filters (Filtermat A, Wallac-Perkin Elmer Life Science, Boston, Miss., USA) using the Harvester 96R (TOMTEC Inc., Hamden, Conn., USA) and [³H]TdR incorporation counted with a liquid scintillation counter (Wallac 1450 Microbeta TM, Wallac-Perkin Elmer Life Science). Results were expressed as average counts per minute of triplicate determinations.

Reverse transcriptase-polymerase chain reaction (RT-PCR) analysis of the expression of the non-germ line configurations of T Cell Receptor Vβ elements. Nucleated cells (1-2×10⁶) were lysed with guanidine isothyocianate (RNAFAST, Molecular System, San Diego, Calif.) and cDNA prepared by reverse transcription with the Moloney reverse transcriptase and random primers (Gibco-BRL, Grand Island, N.Y.). Individual non-germ line configuration of the T Cell Receptor (TCR) Vβ chains were amplified by RT-PCR using a common primer coupled with a primer specific for each of the 22 Vβ family variable elements as described (22). cDNA obtained from RNA extracted from equivalent number of cells was amplified with Pfu DNA polymerase (0.5 U/reaction, Stratagene U.S.A., La Jolla, Calif.) for 35 cycles in a PTC-100 Thermocycler (MJ Research Inc., Watertown, Mass.). Hypoxanthine phosphoribosyl transferase (HPRT) was amplified as control for the quality and the amount of cDNA (not shown). The amplified products were separated by electrophoresis on a 5% polyacrylamide, 10% glycerol non-denaturing gel, which was blotted onto Hybond N⁺ membrane (Amersham-Pharmacia Biotech). The specificity of the amplification was proven by probing with an end-labeled (T4 polynucleotide kinase end labeling kit, Gibco BRL, and γ-³²P-ATP, specific activity=3000 Ci/mmol, Du Pont, Firenze, Italy) oligonucleotides specific for the constant portion of the Vβ elements (23). After hybridization, the filters were washed twice under stringent conditions and exposed to X-ray films (Eastman Kodak Company, Rochester, N.Y.). All the procedures were according to standard protocols (24).

Statistical analysis Statistical analysis was performed with the Mann Whitney Rank Sum test using SigmaStat 2.0 software for Windows (Jandel Corp., San Rafael, Calif., USA).

### RESULTS

Ex vivo Amplification of Total Nucleated Cells and Myeloid Progenitor Cells (CFC) in Serum-Deprived Cultures of CB-LDC Stimulated with Lymphoid Cytokine Combinations.

The fold increase (FI) in total nucleated cells and CFC observed after 10-11 days of serum-deprived cultures of CB-LDC (10⁵ cells/ml) stimulated with lymphoid-specific growth factor combinations is presented herein. Since no differences were observed in the ratio among individual CFC types before and after culture in any of the conditions investigated, only the total CFC number is reported for clarity. The results are compared with those obtained in cultures stimulated with SCF and IL-3, a combination known to stimulate expansion of progenitor cells from neonatal CD34⁺ cells (25-27).

Very few, if any, CB-LDC survived 10-12 days of serum-deprived cultures in the absence of growth factors (19, 25) or when stimulated with only one of the cytokines investigated. SCF and IL-3 induced an increase in the number of total nucleated cells and of CFC in these cultures (average FI=3.6±2.13 and 5.52±2.04, p<0.01, respectively). The presence of SCF+IL-7 increased both the total cell number (FI=6.4±1.17) and the CFC number (FI=8.67±1.5) detected at day 10-12. The increases induced by this growth factor combination were significantly higher (p<0.01) than those observed in the presence of SCF and IL-3. On the other hand, the presence of SCF and IL-4 induced only a modest, although significative (p<0.03), increase (FI=1.9±0.24) in the total number of nucleated cells observed in the culture and a significant increase (FI=10.19±2.0) in CFC numbers, increase that was similar to that sustained by SCF+IL-7. IL-2 had no effect, either alone or in combination with SCF (not shown), on the amplification of CB-LDC under these culture conditions. However, its further addition to culture stimulated with SCF+IL-4 abrogated the increase in CFC number sustained by this growth factor combination (average FI=1.56±0.4).

Lineage Analysis of Mononuclear Cells Expanded in Serum-Deprived Cultures of CB-LDC

The immunophenotype of CB-LDC before and after 10-12 days of culture in the presence of the different growth combinations (SCF+IL-3, SCF+IL-7, SCF+IL-4, SCF+IL-7+IL-2 or SCF+IL-4+IL-2) is presented herein.

Before culture, ≈70% of the CB-LDC expressed a low forward and side scatter characteristic of lymphoid and progenitor cells. Most of the cells present outside this gate were represented either by red blood cells (CD45⁻) or by monocytes (CD33⁺).

After in vitro expansion in the presence of SCF+IL-3, only a minority (12±7%) of the cells fall in the lymphocyte gate (Table 1). The majority of them, expressed the scatter and antigenic profile (CD33⁺) of myeloid cells. In contrast, ≈50% of the cells expanded in the presence of all the lymphoid growth factor combinations analyzed were detected in the lymphocyte gate (Table 1), with the exception of SCF+IL-4 which sustained amplification mostly of myeloid cells (Table 1). Although in percentage, the cells in the lymphoid gate did not increase in any of the culture conditions investigated (68-74% at day 0 vs 44-64% at day 10-12, Table 1), those growth factor combinations that increased the total cell numbers, increased the absolute number of lymphoid cells as well.

The cells present in the lymphocyte gate were identified by FACS immunophenotyping after being stained with the CD7, CD2, CD4, CD8, CD19, CD34 and CD45 antibodies (Table 2). In preliminary experiments, it was verified that all of the cells in the lymphocyte gate expressed CD45 and, therefore, the cells in this gate were not significantly contaminated by erythroid cells. In determining the percent of cells expressing QD4, particular care was paid in setting the gate to exclude possible contamination from CD4⁺ monocytes.

The frequency of cells with the phenotype of T cell precursors (CD7⁺CD2⁻) remained constant when the cultures were stimulated with SCF+IL-7 (FI=2.24±0.83, Table 2), increased when IL-2 was further added to this combination (FI=5.96±3.05, p<0.05), and decreased when the cultures were supplemented with either SCF+IL-4 or SCF+IL-4+IL-2 (FI=0.21±0.07, p<0.001 a 0.52±0.24, respectively) (Table 2).

In contrast to what reported for most of the thymus organ culture systems developed up to now (28-30), the frequency of immature T cells (CD4⁺CD8⁺) did not statistically increase in any of the culture conditions investigated in this paper (Table 2). The only exception was represented by cultures stimulated with SCF+IL-4 where the percentage of CD4⁺CD8⁺ T cells increased from 0.225 (±0.15) at day 0 to 2.25(±1.28) at day 10-12 (FI=14.83±5.22, p<0.05). However, even in this case, the absolute number of the CD4⁺CD8⁺ cells present in the culture remained very low.

A significant increase in the number of mature CD4⁺ (FI=4.72±0.79, p<0.001 ) and CD8⁺ (FI=2.73±1.2, p<0.001) T cells was observed in cultures supplemented with SCF+IL-7. The further addition of IL-2 to these cultures maintained the frequency of CD4⁺ cells at input levels (FI=1.67±0.60) but further increased that of CD8⁺ cells (FI=6.04±0.14, p<0.001). Therefore, addition of IL-2 to cultures supplemented with SCF+IL-7 shifted the predominant T cell phenotype being amplified from CD4⁺ to CD8⁺ (Table 2). In contrast, the frequency of CD4⁺ cells decreased below input values in the presence of SCF+IL-4 or SCF+IL-4+IL-2 (FI=0.64±0.11 and 0.31±0.20, respectively, both p<0.05) while that of CD8⁺ cells was only maintained.

Cells with the CD7⁻CD2⁺ phenotype, that may, at least partially, represent Natural Killer (NK) cells (31), increased in frequency in almost all of the lymphoid growth factor combinations utilized (from 0.7% at day 0 to 5.5% with SCF+IL-7 and 51.0% with SCF+IL-7+IL-2) while the number of cells with a B cell phenotype (CD19⁺) sharply decreased in CB-LDC cultures stimulated with SCF+IL-3 (FI=0.20±0.24), SCF+IL-7 (FI=0.15±0.1), SCF+IL-4 (FI=0.02±0.01, p<0.001) or SCF+IL-4+IL-2 (FI=0.07±0.03, p<0.05), and it was only maintained in the presence of SCF+IL-7+IL-2 (FI=0.92±0.51). These results indicate that the culture conditions investigated here may sustain differentiation of NK cells but do not support proliferation/survival of B-lymphocytes (Table 2).

Approximately 2% of all of the CB-LDC in the lymphocyte gate analyzed before culture were CD34⁺. The frequency of CD34⁺ cells sharply decreased in CB-LDC cultured with any of the growth factor combination investigated. In particular, CD34⁺ cells were below the limit of detection in cultures stimulate with SCF+IL-4 or SCF+IL-4+IL-2, and represented ≈0.1-0.3% of the cells stimulated with SCF+IL-3, SCF+IL-7 or SCF+IL-7+IL-2 (Table 2).

As already reported (21), the cells in the lymphoid gate of CB-LDC differed from those present in the LDC from adult blood (PB-LDC) in CD45RA/RO expression. While PB-LDC contained both CD45RA⁺ and CD45RO⁺ T cells (in a 1:3 ratio), CB-LDC expressed either CD45RA or both CD45RA and CD45RO and very few of them (<2%) expressed only CD45RO. After 10 days of culture, the majority (>90%) of CB T cells obtained in the presence of SCF+IL-7 expressed CD45RA while those obtained in the presence of SCF+IL-7+IL-2 expressed, as PB-LDC, both CD45RA (60%) and CD45RO (30%).

RT-PCR and SSCP Analysis of the TCR β-Chain

To clarify if the increases in T cell numbers observed after 10-12 days in liquid culture of CB-LDC stimulated with SCF+IL-7 were reflected by increases in T cell clonality, the TCR V_{β}-chain repertoire expressed by the cell population before and after culture was compared by RT-PCR and SSCP analysis. In fact, since each T lymphocyte clone expresses a specific TCR gene rearrangement, the complexity of V_{β}-chain fragments amplified from a given T cell population correlates with the number of different T cell clones present in it.

As already reported (32), cord blood samples are heterogeneous in the expression of the rearrangements of the TCR V_{β}-chain genes. Some specimens, present an oligoclonal pattern of expression with some of the V_{β} genes expressed with a limited number of different rearrangements (TCR V_{β} genes from 12 to 20), while others are not expressed at all (6.1, 7, 8, 9, and 10). Other cord blood samples present a polyclonal pattern of expression with multiple different rearrangements detectable for each of the different V_{β} gene.

All of the 22 variable TCR β-chain genes analyzed were found to be expressed by CB-LDC after 10-12 days of culture with SCF+IL-7. This was true even for those TCR V_{β} genes that were not expressed or expressed poorly in the starting CB-LDC population. Although, many of the rearrangements detectable at day 0 were also detected at day 10-11, in many more cases new clonal rearrangements became detectable after culture, while others disappeared.

The expression of TCR V_{β} chain gene fragments had a dynamic pattern. In fact, individual V_{β}-chain fragments detected in culture were highly variable, with new fragments becoming progressively more predominant over time (e.g. the upper band for the V_{β} 4 gene), while others disappearing (e.g. the lower bands detected at day 10 for the V_{β} 5.1 gene) and others still appearing at early time points and disappearing thereafter (e.g. top and lower band for 5.1).

Proliferation of CB-LDC Cultured for 10 Days in the Presence of SCF+IL-7 in Mixed Lymphocite Reaction (MRL).

The proliferation in MRL of T cells obtained from CB-LDC cultured for 10 days in the presence of SCF+IL-7 is presented in Table 3. The data are compared with those obtained with LDC prepared from CB or adult peripheral blood (PB). As already described (21), PB-LDC proliferated very little in the presence of medium alone or of autologous irradiated LDC as stimulators. These cells incorporated high levels of [³H-TdR] in the presence of irradiated allogenic PB-LDC. On the other hand, CB-LDC expressed basal (medium or autologous LDC) proliferation rates significantly higher than those expressed by PB-LDC. They expressed also a high sample to sample variability in their capacity to proliferate in response to irradiated allogenic PB-LDC. For this reason, on average, their proliferation rate in the presence of PB-LDC was not significantly different than that observed under basal conditions. No difference was observed between the proliferation in MRL of CB-LDC before or after culture, with the exception of a significant reduction in proliferation when the cells were stimulated with irradiated autologous LDC.

Liquid Culture of Purified CD4⁺ and CD8⁺ T Cells Stimulated with SCF+IL-7 or SCF+IL-7+IL-2.

To assess the capacity of mature T cells to proliferate under the culture conditions described here, CD4⁺ or CD8⁺ cells were purified by cell sorting from CB-LDC, mixed in a 1:1 ratio and cultured at an initial concentration of 5×10⁶ per ml in the presence of either SCF+IL-7 or SCF+IL-7+IL-2. The total numbers of cells present in the lymphocyte gate remained constant for up to 20 days of culture. All of the cultured cells, present in the lymphocyte gate, were viable (by propidium iodide exclusion, results not shown) and expressed CD3⁺ (T cells). Since a significant proportion of the events were found in the side and forward scatter area characteristic of cellular debris, the number of T cells was likely maintained constant by the balance between proliferation and death. The levels of CD4 and CD8 expression on the cell surface decreased after culture but the CD4⁺/CD8⁺ cell ratio remained similar to the input value (≈1:1) both in cultures stimulated with SCF+IL-7 or SCF+IL-7+IL-2, indicating that no preferential survival/proliferation of CD4⁺ over CD8⁺ cells occurred in any of the two culture conditions.

The TCR V_{β}-chain rearrangement expressed by the purified T cells before and after 10 days of culture is presented herein. This particular specimen expressed at day 0 all of the different V_{β} genes analyzed. Some genes were expressed with a polyclonal pattern (V_{β} 1, 2, 6.1, etc), while others were expressed with an oligoclonal (5.1, 13.2, 15, 16, etc) or even a monoclonal rearrangement pattern (4 and 12). The cultured cells expressed, in general, a TCR repertoire less complex than the original cell population, in agreement with the FACS data which had indicated a high cell mortality in these cultures. Interestingly, cells cultured in SCF+IL-7 or SCF+IL-7+IL-2 expressed different pattern of TCR repertoires. Such variability could be due either to random selection of individual T cell clones or to selective survival of a specific T cell population.

TABLE 1
Percentage of CB-LDC in the lymphoid (low forward and side scatter)
gate at day 0 and at day 10-12 of liquid cultures stimulated with
the indicated growth factor combinations.

| | | | SCF + IL-7 + | | SCF + IL-4 + |
|---|---|---|---|---|---|
| | SCF + IL-3 | SCF + IL-7 | IL-2 | SCF + IL4 | IL-2 |
| day 0 | 75 ± 11 | 71 ± 3 | 74 ± 4 | 68 ± 4 | 68 ± 4 |
| day 10-12 | 12 ± 7* | 44 ± 6 | 57 ± 2 | 27 ± 5* | 64 ± 4 |
| | (n = 4) | (n = 8) | (n = 4) | (n = 4) | (n = 4) |

The results are presented as mean (±SEM) of 4-8 independent experiments, as specified by the number in parenthesis. Percentages statistically different (p<0.05) from the values observed at day 0 are indicated by an asterisk.

TABLE 2
Fold increase in the total number of CD-LDC present in the low side and forward
lymphocyte gate and in the number of cells expressing specific antigenic profiles observed after 10-
12 days of serum-deprived culture stimulated with the indicated growth factor combinations.

| | | | SCF + IL-7 + | | SCF + IL-4 + |
|---|---|---|---|---|---|
| | SCF + IL-3 | SCF + IL-7 | IL-2 | SCF + IL-4 | IL-2 |
| Low forward and | 0.61 ± 0.4 | 2.92 ± 0.6*** | 3.97 ± 0.3*** | 0.75 ± 0.1 | 1.5 ± 0.4 |
| side scatter cells | (n = 4) | (n = 8) | (n = 4) | (n = 4) | (n = 4) |
| CD34⁺ cells | 0.13 ± 0.27* | 0.11 ± 0.06*** | 0.20 ± 0.10*** | b.d.*** | b.d.*** |
| | (n = 4) | (n = 7) | (n = 4) | (n = 4) | (n = 4) |
| CD4⁺/CD8⁻ cells | 0.12 ± 0.13* | 4.72 ± 0.79*** | 1.67 ± 0.60 | 0.64 ± 0.11* | 0.31 ± 0.20* |
| | (n = 4) | (n = 8) | (n = 4) | (n = 4) | (n = 4) |
| CD4⁻/CD8⁺ cells | 0.03 ± 0.05* | 2.73 ± 1.2*** | 6.04 ± 0.14*** | 1.07 ± 0.23 | 1.03 ± 0.20 |
| | (n = 4) | (n = 8) | (n = 4) | (n = 4) | (n = 4) |
| CD4⁺/CD8⁺ cells | b.d.*** | 6.52 ± 2.49 | 10.64 ± 9.78 | 14.83 ± 5.22* | 1.14 ± 0.74 |
| | (n = 4) | (n = 3) | (n = 3) | (n = 4) | (n = 4) |
| CD7⁺/CD2⁻ cells | 0.04 ± 0.08* | 2.24 ± 0.83 | 5.96 ± 3.05* | 0.21 ± 0.07*** | 0.52 ± 0.24 |
| | (n = 4) | (n = 7) | (n = 4) | (n = 4) | (n = 4) |
| CD7⁻/CD2⁺ cells | b.d.*** | 13.08 ± 11.56 | 279.7 ± 70.9*** | 25.01 ± 19.4* | 111.8 ± 91.0** |
| | (n = 4) | (n = 4) | (n = 4) | (n = 4) | (n = 4) |
| CD19⁺ cells | 0.20 ± 0.24 | 0.15 ± 0.10 | 0.92 ± 0.51 | 0.02 ± 0.01*** | 0.07 ± 0.03* |
| | (n = 3) | (n = 3) | (n = 3) | (n = 4) | (n = 4) |

n = number of independent experiments analyzed .
b.d. = below detectable levels.
Significance levels (*p < 0.05 ; **p < 0.01 ; ***p < 0.005) were calculated by Mann Whitney Rank Sum test.

TABLE 3
Proliferative capacity of ex-vivo expanded CB T cells
in primary mixed lymphocyte reaction.

| | | Responders | |
|---|---|---|---|
| Stimulators | PB-LDC | CB-LDC | Cultured CB-LDC* |
| Medium | 3.5 ± 0.8^{a} | 16.1 ± 3.5 | 17.5 ± 9.9 |
| Autologous LDC | 4.8 ± 1.7 | 17.2 ± 0.8 | 8.8 ± 1.5* |
| Allogenic LDC | 45.7 ± 26.1* | 24.0 ± 12.0* | 35.2 ± 30.0 |

^{a}The [3H]TdR incorporation is expressed in cpm (×10³) and the results are presented as mean (±SD) of four separate experiments, each one performed in triplicate. Asterisks indicate incorporations statistically different (p < 0.05) form those observed with medium alone.

### REFERENCES

1. Gluckman E. Current status of umbilical cord blood hematopoietic stem cell transplantation. Exp Hematol 2000; 28 (11): 1197-205.
2. Rubinstein P, Carrier C, Scaradavou A et al. Outcomes among 562 recipients of placental-blood transplants from unrelated donors. N Engl J Med 1998; 339: 1565-77
3. Hassan J, Reen D J. Cord blood CD4+CD45RA+T cells achieved a lower magnitude of activation when compared with their adult counterparts. Immunology 1997; 90: 397-401
4. Harris D T, Schumacher M J, Locascio J et al. Phenotypic and functional immaturity of human umbilical cord blood T lymphocytes. Proc Natl Acad Sci USA 1992; 89: 10006-10
5. Risdon G, Gaddy J, Horie et al. Alloantigen priming induces a state of unresponsiveness in human umbilical cord blood T cells. Proc Natl Acad Sci USA 1995; 92: 2413-17
6. Kolb H J, Mittermuller J, Clemm C et al. Donor leukocyte transfusion for treatment of recurrent chronic myelogeneous leukemia in marrow transplant patients. Blood 1990; 76: 2462-65
7. Dazzi F, Szydlo R M, Goldman J M. Donor lymphocyte infusion for relapse of chronic myeloid leukemia after allogeneic stem cell transplant: Where we now stand. Exp Hematol 1999; 27: 1477-86
8. Gross S, Pyatt D W, Shpall E J et al. Ex vivo expansion of CD3-cells from cord blood for use as donor lymphocyte infusions. Exp Hematol 2000; 28 (suppl.1): 89 (abstr. 185)
9. Carlens S, Gilljam M, Remberger M et al. Ex vivo T lymphocyte expansion for retroviral transduction: Influence of serum-free media on variations in cell expansion rate and lymphcyte subset distribution. Exp Hematol 2000; 28: 1137-46.
10. Skea D, Chang N H, Hedge R et al. Large ex vivo expansion of human umbilical cord blood CD4+ and CD8+ T cells. J Hematother 1999; 8: 129-39.
11. Azuma H, Yamada Y, Shibuya-Fujiwara N et al. Functional evaluation of ex vivo expanded cord blood lymphocytes: Possible use for adoptive cellular immunotherapy. Exp Hematol 2002; 30: 346-51
12. Bonini C, Ferrari G, Verzeletti S et al. HSV-TK gene transfer into donor lymphocytes for control of allogeneic graft-versus-leukemia. Science 1997; 276: 1719-24.
13. Peschon J J, Morrissey P J, Grabstein K H et al. Early Lymphocyte expansion in severely impaired in interleukin 7 receptor-deficient mice. J Exp Med 1994; 180: 1955-60
14. Hofmeister R, Khaled A R, Benbernou N et al. Interleukin-7: physiological roles and mechanisms of action. Cytokine Growth Factor Rev 1999; 10: 41-60
15. Sadlack B, Kuhn R, Schorle H et al. Development and proliferation in lymphocytes in mice deficient for both interleukins-2 and -4. Eur J Immunol 1994; 24: 281-84
16. Kuhn R, Rajevsky K, Muller W. Generation and analysis of interleukin-4 deficient mice. Science 1991; 254: 707-10
17. Gluckman E, Rocha V, Boyer-Chammard A et al. Outcome of cord-blood transplantation from related and unrelated donors. N Engl J Med 1997; 337(6): 373-81
19. Forte L, Migliaccio G, Sanchez M, Migliaccio A R et al. Effects of cell banking manipulations on ex vivo amplification of umbilical cord blood. Ann 1st Super Sanità 2000; 36: 333-342.
19. Sanchez M, Alfani E, Visconti G et al. Thymus-independent T-cell differentiation in vitro. Br J Haematol 1998; 103: 1198-205
20. Migliaccio G, Migliaccio A R, Adamson J W. In vitro differentiation of human granulocyte/macrophage and erythroid progenitors: comparative analysis of the influence of recombinant human erythropoietin, G-CSF, GM-CSF, and IL-3 in serum-supplemented and serum-deprived cultures. Blood 1988; 72 (1): 248-256.
21. Roncarolo M G, Bigler M, Ciuti E el al. Immune responses by cord blood cells. Blood Cells 1994, 20:573-586.
22. Choi Y, Kotzin B, Herron L et al. Interaction of Staphylococcus aureus toxin “superantigens” with human T-cells. Proc. Natl. Acad. Sci. U.S.A. 1989; 86: 8941-45
23. Yamamoto K, Masuko-Hongo K, Tanaka A et al. Establishment and application of a novel T cell clonality analysis using single-strand conformation polymorphim of T cell receptor messenger signals. H Immunol 1996; 48: 23-31
24. Maniatis T, Fritsch E F and Sambrook J. Molecular Cloning: A Laboratory Manual, 2nd ed. 1989. Cold Spring Harbor Lab. Press. NY
25. Migliaccio G, Migliaccio A R, Druzin M L et al. Long-term generation of colony-forming cells in liquid culture of CD34⁺ cord blood cells in the presence of recombinant human stem cell factor. Blood 1992; 79 (10):2620-2627
26. Migliaccio A R, Migliaccio G, Adamson J W. Expansion of human neonatal progenitor cells in vitro under serum-deprived conditions. Blood Cells 1994; 20 (2-3): 424-428.
27. Rossmanith T, Schroder B, Bug G et al. Interleukin 3 improves the ex vivo expansion of primitive human cord blood progenitor cells and maintains the engraftment potential of scid repopulating cells. Stem Cells 2001; 19 (4): 313-320.
28. Freedman A R, Zhu H, Levine J D et al. Generation of human lymphocytes from bone marrow CD34⁺ cells in vitro. Nat Med 1996; 2: 46-51
29. Plum J, De Smedt M, Defresne M-P et al. Interleukin-7 is a critical growth factor in early human T-cell development. Blood 1996; 88: 4239-45
30. Hirayama F, Aiba Y, Ikebuchi K et al. Differentiation in culture of murine primitive lymphohematopoietic progenitors toward T-cell lineage. Blood 1999; 93: 4187-95.
31. Lima M, Almeida J, dos Ajos Teixeira M et al. The “ex vivo” pattern of CD2/CD7, CD57/CD11c, CD387CD11b, CD45RA/CD45RO, and CD11a/HLA-DR expression identify acute/early and chronic/late NK-cell activation state Bloods Cells Mol Dis 2002; 28 (2): 181-90.
32. Alfani E, Migliaccio A R, Sanchez, M et al. Characterization of the T cell receptor repertoire of neonatal T cells by RT-PCR and single strand comformation polymorphism analysis. Bone Marrow Tranplant. 2000; 26: 83-89
33. Brandt J, Briddell R A, Srour E F et al. Role of c-kit ligand in the expansion of human hematopoietic progenitor cells. Blood 1992; 79 (3): 634-641,.
34. Parkin J, Cohen B An overview of the immune system. The Lancet 2001; 357: 1777-89
35. Von Freeden-Jeffry U, Vieira P, Lucian L A et al. Lymphopenia in interleukin (IL)-7 gene-delete mice identifies IL-7 as a non-redundant cytokine. J Exp Med 1995; 181 (4): 1519-26
36. Wiles M V, Ruiz P, Imhof B A. Interleukin-7 expression during mouse thymus development. Eur J Immunol 1992; 22: 1037-42.
37. Swain S L, Bradley L M, Croft M et al. Helper T-cell subsets: phenotype, function and the role of lymphokines in their development. Immunol Rev 1991; 123: 115-44.
38. Spellberg B, Edwards J E Jr. Type 1/Type 2 immunity in infectious diseases. Clin Infect Dis 2001; 32 (1):76-102.
39. Chervenak R, Dempsey D, Soloff R S, Smithson G. In vitro growth of bone marrow resident T cell precursors supported by mast cell growth factor and IL-3. J. Immunol 1992; 149: 2851-2856
40. Yeoman H, Clark D R, De Luca D. Development of CD4 and CD8 single positive T cells in human thymus organ culture: IL-7 promotes human T cell production by supporting immature T cells. Dev Comp Immunol 1996; 20: 241-63
41. Tagoh H, Kishi H, Okumura A et al. Induction of recombination activating gene expression in a human lymphoid progenitor cell line: requirement of two separate signals from stromal cells and cytokines. Blood 1996; 88: 4463-73
42. Garcia-Ojeda M E, Dejbakhsh-Jones S, Weissman I L, Strober S. An alternate pathway for T cell development supported by the bone marrow microenvironment: recapitulation of thymic maturation. J Exp Med 1998; 187 (11): 1813-23
43. Takeshita T, Asao H, Ohtani K et al. Cloning of the gamma chain of the human IL-2 receptor. Science 1992; 257: 379-82.
44. Noguchi M, Nakamura Y, Russell S M et al. Interleukin-2 receptor gamma chain: a functional component of the interleukin-7 receptor. Science 1993; 262: 1877-80
45. Kondo M, Takeshita T, Ishii N et al. Sharing of the interleukin-2 (IL-2) receptor gamma chain between receptor for IL-2 and IL-4. Science 1993; 262: 1874-77
46. Russell S M, Keegan A D, Harada N et al. Interleukin-2 receptor gamma chain: a functional component of the interleukin-4 receptor. Science 1993; 262: 1880-83

1. A method of ex vivo amplification of neonatal T cells comprising the steps of:
(a) obtaining light density mononuclear cells from a sample of umbilical cord blood; and
(b) stimulating T cell growth by incubating said light density mononuclear cells in a serum deprived culture medium comprising a combination of cytokines comprising stem cell factor and interleukin-7, thereby obtaining selective amplification of T cells.

2. The method of claim 1, wherein each of stem cell factor and interleukin-7 is at a concentration of about 10 ng/ml.

3. The method of claim 1, wherein said light density mononuclear cells are incubated in said serum deprived culture medium for up to 12 days.

4. The method of claim 1, wherein preferential amplification of CD4⁺ over CD8⁺ T cell subsets is obtained.

5. The method of claim 1, wherein said combination of cytokines further comprises interleukin-2.

6. The method of claim 2, wherein each of stem cell factor, interleukin-7 and interleukin-2 is at a concentration of about 10 ng/ml.

7. The method of claim 2, wherein said light density mononuclear cells are incubated in said serum deprived culture medium for up to 12 days.

8. The method of claim 2, wherein preferential amplification of CD8⁺ over CD4⁺ T cell subsets is obtained.
